SEQUENCE LISTING (16 sequences)

SEQ ID NO: 1 - protein, 9 aa, Artificial Sequence
  Synthetic- Peptide linker
NLVPMVATV

SEQ ID NO: 2 - protein, 10 aa, Artificial Sequence
  Synthetic- Mart-1 peptide
ELAGIGILTV

SEQ ID NO: 3 - protein, 5 aa, Artificial Sequence
  Synthetic- Peptide linker
EAAAK

SEQ ID NO: 4 - DNA, 20 bp, Artificial Sequence
  Synthetic- B2M sg2
actcacgctggatagcctcc

SEQ ID NO: 5 - DNA, 20 bp, Artificial Sequence
  Synthetic- B2M sg4
ggccacggagcgagacatct

SEQ ID NO: 6 - DNA, 101 bp, Homo sapiens
ttcgggccgagatgtctcgctccgtggccttagctgtgctcgcgctactctctctctttctggcctggaggctatccagcgtgagtctctcctaccctccc

SEQ ID NO: 7 - DNA, 101 bp, Homo sapiens
aagcccggctctacagagcgaggcaccggaatcgacacgagcgcgatgagagagagaaagaccggacctccgataggtcgcactcagagaggatgggaggg

SEQ ID NO: 8 - protein, 22 aa, Homo sapiens
MSRSVALAVLALLSLSGLEAIQ

SEQ ID NO: 9 - DNA, 20 bp, Artificial Sequence
  Synthetic- B2M sg2 reverse complement
cctccgataggtcgcactca

SEQ ID NO: 10 - DNA, 75 bp, Homo sapiens
ggcattcctgaagctgacagcattcgggccgagatgtctcgctccgtggccttagctgtgctcgcgctactctct

SEQ ID NO: 11 - DNA, 75 bp, Homo sapiens
ccgtaaggacttcgactgtcgtaagcccggctctacagagcgaggcaccggaatcgacacgagcgcgatgagaga

SEQ ID NO: 12 - DNA, 20 bp, Artificial Sequence
  Synthetic- B2M sg4 reverse complement
tctacagagcgaggcaccgg

SEQ ID NO: 13 - DNA, 152 bp, Homo sapiens
ggaccagagcgggagggtaggagagactcacgctggatggatcctcctcctccggatcctcctcctccggatcctcctcctcccacggtcaggatgccgatgccggccagctcagcctccaggccagaaagagagagtagcgcgagcacagc

SEQ ID NO: 14 - DNA, 152 bp, Homo sapiens
cctggtctcgccctcccatcctctctgagtgcgacctacctaggaggaggaggcctaggaggaggaggcctaggaggaggagggtgccagtcctacggctacggccggtcgagtcggaggtccggtctttctctctcatcgcgctcgtgtcg

SEQ ID NO: 15 - DNA, 38 bp, Homo sapiens
ggaccagagcgggagggtaggagagactcacgctggat

SEQ ID NO: 16 - DNA, 39 bp, Homo sapiens
agcctccaggccagaaagagagagtagcgcgagcacagc

(19) United States
(12) Patent Application Publication
(10) Pub. No.: US 2024/0181054 A1
(43) Pub. Date: Jun. 6, 2024

(54) Genetically Modified Cells Expressing Antigen-Containing Fusion Proteins and Uses Thereof

(71) Applicant: Synthego Corporation, Redwood City, CA (US)

(72) Inventors: Robert DEANS, Riverside, CA (US); Travis MAURES, La Jolla, CA (US); Yueting ZHENG, Fremont, CA (US); Jared Carlson STEVERMER, Burlingame, CA (US); Monique DAO, Mountain View, CA (US); Phillip BALZANO, Redwood City, CA (US); Rebecca NUGENT, Redwood City, CA (US)

(21) Appl. No.: 18/550,205

(22) PCT Filed: Mar. 14, 2022
(86) PCT No.: PCT/US2022/020225

§ 371 Date: Sep. 12, 2023

Related U.S. Application Data
(60) Provisional application No. 63/160,382, filed on Mar. 12, 2021.

(51) Int. Cl. A61K 39/00; C07K 14/74; C12N 5/0781; C12N 9/22; C12N 15/11; C12N 15/90

(52) U.S. Cl. CPC A61K 39/4622; A61K 39/4612; A61K 39/4634; A61K 39/464491; C07K 14/70539; C12N 5/0635; C12N 9/22; C12N 15/11; C12N 15/907; C07K 2319/40; C12N 2310/20; C12N 2510/00; C12N 2800/80

(57) ABSTRACT

The application relates to biological components, methods, systems, and kits for modulating immune responses. The disclosed biological components include genetically modified cells comprising an inserted exogenous sequence in a major histocompatibility complex (MHC)-associated gene. The inserted exogenous sequence encodes a peptide and the genetically modified cells express a fusion protein comprising the peptide and at least a portion of the polypeptide encoded by the MHC-associated gene to form a modified MHC complex. The genetically modified cells may present the peptide as an antigen associated with the MHC complex and may be utilized in methods for modulating T cell activity, inducing an immune response, and inducing a tolerogenic response. As such, the disclosed biological components, methods, systems, and kits may be utilized in order to treat and/or prevent a disease or disorder in a subject in need thereof and to screen and validate clinically relevant antigens.

### CROSS-REFERENCE TO RELATED PATENT APPLICATIONS

The present application claims the benefit of priority under 35 U.S.C. § 119(e) to U.S. Provisional Application No. 63/160,382, filed on Mar. 12, 2021, the contents of which are incorporated herein by reference in their entirety.

### SEQUENCE LISTING

A Sequence Listing accompanies this application and is submitted as an ASCII text file of the sequence listing named “174774_00106_ST25.txt” which is 3,845 bytes in size and was created on Mar. 11, 2022. The sequence listing is electronically submitted via EFS-Web with the application and is incorporated herein by reference in its entirety.

### BACKGROUND

The major histocompatibility complex (MHC) is a large genetic locus in vertebrates that contains a set of polymorphic genes that encode for cell surface proteins that are utilized for adaptive immunity. The cell surface proteins are called MHC molecules and include human leukocyte antigens (HLAs) and beta-2 microglobulin (B2M). MHC molecules are utilized by cells as a framework to display internally processed protein fragments as antigens on the cell surface to immune cells such as T cells. Displayed as such, the antigens may induce an immune response in T cells exposed to the MHC-antigen complex.

Two classes MHC molecules exist, Class I MHC molecules and Class II MHC molecules. Class I MHC molecules are expressed on somatic cells and are used as recognition elements for T cells in immune surveillance. In order for a T cell to recognize an antigen as “non-self,” the antigen must be displayed by Class I MHC molecules. This is the core decision for self versus non-self recognition in the immune system. Tumor antigens and autoimmune antigens are considered non-self in the context of disease etiology. Class II MHC molecules are expressed on immune regulatory cells or cells involved in immune homeostasis and inflammation. Dendritic cells are an example of immune regulatory cells which express class II MHC and stimulate a T cell response. Dendritic cells can regulate an effector T cell response such as tumor killing, or tolerize and suppress an immune response based on pathogen or disease associated paracrine signals.

There exists a need in the field for novel components and methods for modulating immune responses so as to screen and to validate clinically relevant antigens, and to treat and/or to prevent a disease or disorder in a subject.

### SUMMARY

Disclosed herein are components, methods, systems, and kits which may be utilized for modulating immune responses. The disclosed biological components include genetically modified cells comprising an inserted exogenous sequence in a major histocompatibility complex (MHC)-associated gene. The inserted exogenous sequence encodes a peptide and the genetically modified cells express a fusion protein comprising the peptide and at least a portion of the polypeptide encoded by the MHC-associated gene to form a modified MHC complex. The genetically modified cells may present the peptide as an antigen associated with the MHC complex and may be utilized in methods for modulating T cell activity. The disclosed methods may be performed in order to induce an immune response in a subject in need thereof and in methods for inducing a tolerogenic response in need thereof. As such, the disclosed biological components, methods, systems, and kits may be utilized in order to treat and/or prevent a disease or disorder in a subject in need thereof by modulating an immune response in the subject and to screen and validate clinically relevant antigens.

In some embodiments, the disclosed components and methods may be utilized for preparing genetically modified cells which have been modified to express an exogenous peptide. The disclosed components may include components for engineering cells and the disclosed components may include genetically modified cells. The genetically modified cells may present the peptide as an antigen associated with the MHC complex and may be utilized in methods for modulating T cell activity in methods for inducing an immune response and in methods for inducing a tolerogenic response. In some embodiments, the disclosed components and methods may be utilized for modulating an immune response, such as an adaptive immune response.

In some embodiments, the disclosed components and methods may be utilized to engineer cells having a genetically modified class I MHC-associated loci which expresses an exogenous peptide that may function as an antigen. The disclosed components and methods may be utilized to screen and validate exogenous peptides that may function as clinically relevant antigens, and further the disclosed components and methods may be utilized to treat and/or to prevent a disease or disorder in a subject associated with an antigen.

In some embodiments, the disclosed components and methods may be utilized to express an exogenous peptide as an antigen, and as such, the disclosed technology which utilizes the disclosed components and methods may be referred to as “WRITE” (i.e., “write antigen”), whereby gene editing introduces genetic information in the form of knocking in exogenous peptides directly into MHC-associated molecules for T cell recognition and use in adaptive immunotherapy. WRITE may comprise a genome engineered immune modulatory platform that moves from identifying an antigen to expressing the antigen in one biologic step through gene editing of cells such as antigen presenting cells. WRITE may utilize gene editing technologies, such as CRISPR, ZFN, TALEN, or vectors to directly edit endogenous MHC molecules and create and/or express fusion proteins comprising the antigens. By expressing the antigens in the context of fusion proteins, WRITE may bypass antigen processing, transport, and display. Based on insights into the structure of MHC molecules, the inventors have devised a method and rationale to directly incorporate information for antigen specificity into the MHC molecule itself. The ability to control design elements at class I MHC molecules can be read into a high throughput library screens for T cell recognition relevant to disease. These design elements can be genetically inserted at MHC loci in cells, which may include antigen presenting cells (APCs), and may be used to modulate immune responses.

### BRIEF DESCRIPTION OF THE FIGURES

FIG. 1. Editing of MHC-associated genes and insertion of exogenous sequence encoding an antigenic peptide. (A) An exogenous sequence encoding a peptide NLVPMVATV (“NLV peptide”) and a linker was knocked-in between the beta-2 microglobulin (B2M) signaling peptide and the mature B2M protein. (B) The NLV peptide (circle) is fused to the N-terminus of B2M by a (G₄S)₃ linker (hatched bar), where the peptide-linker sequence may be referred to as NLVPMVATV(SEQ ID NO: 1)(G₄S)₃, and the peptide is presented in the peptide-binding cleft of the human leukocyte antigen (HLA).

FIG. 2. Insertion of exogenous sequence encoding a fusion protein, and replacement of endogenous MHC-associated genes. (A) An exogenous sequence encoding a NLV peptide single chain trimer was knocked-in at the 5′ end of the B2M coding region, abolishing expression of the endogenous B2M. The encoded NLV peptide (circle) is fused to the N terminus of B2M by a (G₄S)₃ linker (hatched bar), and the HLA-A2 polypeptide is fused to the C terminus of B2M by a (G₄S)₄ linker (hatched bar), forming the NLV peptide single chain trimer (SCT). The fusion protein further includes a C-terminal P2A sequence and a Q8 tag. (B) The single chain trimer presents the NLV peptide within the peptide binding cleft.

FIG. 3. Engineering of lymphoblastoid cell lines (LCLs) to express NLV peptide and FACS analysis. (A) FACS analysis of class II MHC, transactivator (CIITA) expression in LCL lines in which the CIITA gene was knocked-out. The CIITA gene was knocked-out from three different HLA-A2+ LCL lines (i.e., LCL-4, LCL-5, and LCL-6). The parent lines are represented by the peak on the right, and the CIITA⁻ lines are represented by the peak on the left. (B) FACS analysis of HLA-A2 and B2M expression in CIITA⁻ LCL lines and NLV peptide knock-in LCL lines. (C) FACS analysis of expression of the Q8 tag, B2M, and HLA-A2 in CIITA⁻ LCL lines and SCT knock-in LCL lines.

FIG. 4. T cell activation by edited antigen presenting cells (APCs). (A) the HLA-types of two healthy donors (i.e., Donor 1 and Donor 2) and two LCL lines (i.e., LCL-4 and LCL-6) were compared. The class I HLA of Donor 1 and Donor 2 were matched with LCL-4 and LCL-6, respectively. (B) IFNγ ELISPOT was used to assay activation of NLV peptide-specific CTL lines by the edited LCL-4 and LCL-6 lines utilized as APCs. NLV-specific CTL lines were generated from each of Donor 1 and Donor 2. The CTL lines then were cultured in the presence of various class I HLA-matched LCLs. Row 1, no stimuli, negative control; Row 2, PHA, positive control; Row 3, CIITA-negative LCL; Row 4, CIITA-negative LCL pulsed with the NLV peptide; Row 5, CIITA-negative LCL expressing the NLV peptide fused to B2M; and Row 6, CIITA-negative LCL expressing the NLV single chain trimer.

FIG. 5. Insertion of antigen peptide elicits antigen-specific CD8+ T cell responses. a) Lymphoblast cell line cells (LCLs) were engineered to express the melanoma antigen MART-1 peptide (ELAGIGILTV (SEQ ID NO: 2)) tethered to the beta-2 microglobulin (B2M) subunit of HLA class I. Edited cells were then co-cultured overnight with commercially obtained CD8+ cytotoxic T cells specifically reactive to the ELAGIGILTV (SEQ ID NO: 2) peptide at defined effector:target (E:T) ratios. b) MART-1 specific CD8+ T cells were mixed at three different E:T ratios with pre-stained HLA-matched LCL either pulsed with MART-1 peptide (pLCL) or engineered to express the MART-1 epitope in the context of HLA-A*0201 (KI MART-1LCL). Cells were co-cultured overnight before flow cytometry analysis. Bars represent mean +/− SE from duplicate wells. Data are of a single representative experiment (n=3). Significance defined by paired Student's t test—* , P<0.05; ** , P<0.01; *** , P<0.001; ***. c) Increase of percentage of surface-exposed CD107a (LAMP1) positive CD8+ T cells indicates increase in CD8+ T cell activation, target-specific recognition, and killing. T Cells were co-cultured overnight with targets before staining flow cytometry analysis. Bars represent mean +/− SE from duplicate wells. Data are of a single representative experiment (n=3). Significance defined by paired Student's t test—* , P<0.05; ** , P<0.01; *** , P<0.001.

### DETAILED DESCRIPTION

The present invention is described herein using several definitions, as set forth below and throughout the application.

Unless otherwise specified or indicated by context, the terms “a”, “an”, and “the” mean “one or more.” For example, “a component,” “a composition,” “a system,” “a kit,” “a method,” “a protein,” “a vector,” “a domain,” “a binding site,” “an RNA,” “a cell,” “a gene,” “an insertion,” “an antigen,” should be interpreted to mean “one or more components,” “one or more compositions,” “one or more systems,” “one or more kits,” “one or more methods,” “one or more proteins,” “one or more vectors,” “one or more domains,” “one or more binding sites,” “one or more RNAs,” “one or more cells,” one or more genes,” “one or more insertions,” and “one or more antigens,” respectively.

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, a subject in need thereof may include a subject having or at risk for developing a disease or disorder that may be treated and/or prevented by modulating an immune response in the subject. As disclosed herein, “modulation” may include induction and/or enhancement of an immune response in a subject. As disclosed herein, “modulation” also may include reduction or elimination of an immune response and/or induction of tolerance in a subject. A subject may include a human subject or a non-human subject (e.g., dogs, cats, horses, cows, pigs, and the like).

### Polynucleotides and Uses Thereof

The disclosed subject matter relates to polynucleotides and the uses thereof. As used herein, the terms “polynucleotide,” “polynucleotide sequence,” “nucleic acid,” and “nucleic acid sequence” refer to a nucleotide, oligonucleotide, polynucleotide (which terms may be used interchangeably), or any fragment thereof. These phrases also refer to DNA or RNA of genomic, natural, or synthetic origin (which may be single-stranded or double-stranded and may represent the sense or the antisense strand).

The terms “nucleic acid” and “oligonucleotide,” as used herein, may refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA.

Polynucleotide sequence may exhibit homology or percentage identify to a reference polynucleotide sequence. Regarding polynucleotide sequences, the terms “percent identity” and “% identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides.

Regarding polynucleotide sequences, a “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

The terms “target,” “target sequence,” “target region,” and “target nucleic acid,” as used herein, are synonymous and may refer to a region or sequence of a nucleic acid which is to be hybridized and/or bound by another nucleic acid (e.g., a target sequence that is targeted for recombination).

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, New York; Wetmur, 1991, Critical Review in Biochem. and Mol. Biol. 26(3/4):227-259; and Owczarzy et al., 2008, Biochemistry, 47: 5336-5353, which are incorporated herein by reference).

As used herein, a polynucleotide sequence is “specific,” for a target sequence if, when used in an amplification reaction under sufficiently stringent conditions, the primer hybridizes primarily to the target nucleic acid. Typically, a polynucleotide sequence is specific for a target sequence if the stability between the polynucleotide sequence and the target is greater than the stability of a duplex formed between the polynucleotide sequence and any other sequence found in the sample. One of skill in the art will recognize that various factors, such as salt conditions as well as base composition of the primer and the location of the mismatches, will affect the specificity of the polynucleotide sequence, and that routine experimental confirmation of the polynucleotide sequence specificity will be needed in many cases. Hybridization conditions can be chosen under which the polynucleotide sequence can form stable duplexes only with a target sequence. Thus, the use of target-specific polynucleotide sequence under suitably stringent amplification conditions enables the target sequence for hybridization and recombination.

As used herein, “an engineered transcription template” or “an engineered expression template” refers to a non-naturally occurring nucleic acid that serves as substrate for transcribing at least one RNA. As used herein, “expression template” and “transcription template” have the same meaning and are used interchangeably. Engineered include nucleic acids composed of DNA or RNA.

The polynucleotides disclosed herein may be expressed from a promoter. The term “promoter” refers to a cis-acting DNA sequence that directs RNA polymerase and other trans-acting transcription factors to initiate RNA transcription from the DNA template that includes the cis-acting DNA sequence.

The polynucleotide sequences contemplated herein may be present in expression vectors. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise an exogenous promoter operably linked to a polynucleotide that encodes a protein. An “exogenous promoter” refers to a promoter that is not the native or endogenous promoter for the protein or RNA that is being expressed.

As used herein, “expression” refers to the process by which a polynucleotide is transcribed from a DNA template (such as into mRNA or another RNA transcript) and/or the process by which a transcribed mRNA is subsequently translated into peptides, polypeptides, or proteins. Transcripts and encoded polypeptides may be collectively referred to as “gene products.”

The term “vector” refers to some means by which nucleic acid (e.g., DNA) can be introduced into a host organism or host tissue. There are various types of vectors including plasmid vector and viral vectors. As used herein, a “vector” may refer to a recombinant nucleic acid that has been engineered to express an exogenous polypeptide (e.g., the fusion proteins disclosed herein). The recombinant nucleic acid typically includes cis-acting elements for expression of the exogenous polypeptide.

In the methods contemplated herein, a host cell may be transiently or non-transiently transfected (i.e., stably transfected) with one or more vectors described herein. A cell transfected with one or more vectors described herein may be used to establish a new cell line comprising one or more vector-derived sequences. In the methods contemplated herein, a cell may be transiently transfected with the components of a system as described herein (such as by transient transfection of one or more vectors), and modified through the activity of a complex, in order to establish a new cell line comprising cells containing the modification but lacking any other exogenous sequence.

“Transformation” or “transfection” describes a process by which exogenous nucleic acid (e.g., DNA or RNA) is introduced into a recipient cell. Transformation or transfection may occur under natural or artificial conditions according to various methods well known in the art and may rely on any known method for the insertion of foreign nucleic acid sequences into a cell. The method for transformation or transfection is selected based on the type of host cell being transformed and may include, but is not limited to, viral infection or non-viral delivery. Methods of non-viral delivery of nucleic acids include lipofection, nucleofection, microinjection, electroporation, heat shock, particle bombardment, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam.™. and Lipofectin.™.). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration).

### Peptides, Polypeptides, and Proteins

The disclosed subject matter relates to peptides and polypeptides which may include fusion polypeptides. As used herein, the terms “peptide” or “polypeptide” or “protein” may be used interchangeable to refer to a polymer of amino acids. Typically, a “polypeptide” or “protein” is defined as a longer polymer of amino acids, of a length typically of greater than 50, 60, 70, 80, 90, or 100 amino acids. A “peptide” typically is defined as a short polymer of amino acids, of a length typically of 50, 40, 30, 20 or less amino acids.

A “polypeptide,” “protein,” or “peptide” as contemplated herein typically comprises a polymer of coding amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). The proteins disclosed herein may include “wild type” proteins and variants, mutants, and derivatives thereof. As used herein the term “wild type” is a term of the art understood by skilled persons and means the typical form of an organism, strain, gene or characteristic as it occurs in nature as distinguished from mutant or variant forms. As used herein, a “variant, “mutant,” or “derivative” refers to a protein molecule having an amino acid sequence that differs from a reference protein or polypeptide molecule. A variant or mutant may have one or more insertions, deletions, or substitutions of an amino acid residue relative to a reference molecule. A variant or mutant may include a fragment of a reference molecule. For example, a mutant or variant molecule may have one or more insertions, deletions, or substitution of at least one amino acid residue relative to a reference polypeptide.

A “deletion” refers to a change in the amino acid sequence that results in the absence of one or more amino acid residues. A deletion may remove at least 1, 2, 3, 4, 5, 10, 20, 50, 100, 200, or more amino acids residues. A deletion may include an internal deletion and/or a terminal deletion (e.g., an N-terminal truncation, a C-terminal truncation or both of a reference polypeptide). A “variant,” “mutant,” or “derivative” of a reference polypeptide sequence may include a deletion relative to the reference polypeptide sequence.

A “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 25, 30, 40, 50, 60, 70, 80, 90, 100, 150, 250, or 500 contiguous amino acid residues of a reference polypeptide. Fragments may be preferentially selected from certain regions of a molecule. The term “at least a fragment” encompasses the full-length polypeptide. A fragment may include an N-terminal truncation, a C-terminal truncation, or both truncations relative to the full-length protein. A “variant,” “mutant,” or “derivative” of a reference polypeptide sequence may include a fragment of the reference polypeptide sequence.

The words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, 200, or more amino acid residues. A “variant,” “mutant,” or “derivative” of a reference polypeptide sequence may include an insertion or addition relative to the reference polypeptide sequence. A variant of a protein may have N-terminal insertions, C-terminal insertions, internal insertions, or any combination of N-terminal insertions, C-terminal insertions, and internal insertions.

The word “fusion” refers to a polypeptide sequence comprising an exogenous amino acid sequence fused to a native amino acid sequence. Fusion proteins include proteins comprising at least a portion of the amino acid sequence of a major histocompatibility complex (MHC)-associated protein fused to an exogenous amino acid sequence, either directly or indirectly via an intervening linking amino acid sequence. The exogenous sequence may be fused at the N-terminus of the native amino acid sequence, at the C-terminus of the native amino acid sequence, or internally within the native amino acid sequence such that the fusion protein comprising an N-terminal portion of the native amino acid sequence, the exogenous amino acid sequence, and a C-terminal portion of the native amino acid sequence. Two polypeptide sequences may be fused directly without any intervening amino acid sequence and/or two polypeptide sequences may be fused via a linker as known in the art.

Regarding proteins, the phrases “percent identity” and “% identity,” refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

Regarding proteins, percent identity may be measured over the length of an entire defined polypeptide sequence, for example, over the length of a fragment taken from a larger, defined polypeptide sequence, for instance, a fragment of at least 15, at least 20, at least 30, at least 40, at least 50, at least 70 or at least 150 contiguous residues.

The disclosed proteins, mutants, variants, or described herein may have one or more functional or biological activities exhibited by a reference polypeptide (e.g., one or more functional or biological activities exhibited by wild-type protein).

In some embodiments of the disclosed compositions, systems, kits, and methods, the components may be substantially isolated or purified. The term “substantially isolated or purified” refers to components that are removed from their natural environment, and are at least 60% free, preferably at least 75% free, and more preferably at least 90% free, even more preferably at least 95% free from other components with which they are naturally associated.

### Genetically Modified Cells Comprising an Inserted Exogenous Sequence Encoding an Antigenic Peptide in a Major Histocompatibility Complex (MHC)-Associated Gene and Expressing Antigen-Containing Fusion Proteins

The disclosed subject matter relates to genetically modified cells comprising an inserted exogenous sequence in a major histocompatibility complex (MHC)-associated gene. The inserted exogenous sequence encodes a peptide and the genetically modified cells express a fusion protein comprising the peptide and at least a portion of the polypeptide encoded by the MHC-associated gene. The genetically modified cells may present the peptide as an antigen associated with the MHC complex. As such, the genetically modified cells may be utilized in methods, systems, and kits for modulating T cell activity in a subject in need thereof, and in methods for treating diseases and disorders in a subject in need thereof and to screen and validate clinically relevant antigens.

In some embodiments, the disclosed methods may be performed in order to induce and/or enhance an immune response in a subject in need thereof, thereby treating and/or preventing a disease or disorder in the subject. Diseases and disorders that may be treated and/or prevented by an immune response induced by the disclosed methods may include, but are not limited to, cell proliferative diseases and disorders (e.g., cancers) and microbial infections (e.g., viral infections, bacterial infections, fungal infections and the like). The peptide may be an antigen associated with a disease or disorder accordingly (e.g., a neoantigen associated with a cancer, an antigen associated with a virus, bacterial, or fungus, or a self-antigen that is associated with an autoimmune disease).

Immune responses induced by the disclosed methods may include T cell responses. In some embodiments, the disclosed methods may be performed in order to activate T cells in a subject in need thereof. “T cell activation” may be assessed using methods known in the art, including but not limited to, enzyme-linked immunospot (ELISPOT) in order to measure T cell activation by production of cytokines that are associated with activation.

In some embodiments, the disclosed methods may be performed in order to reduce and/or eliminate an immune response in a subject or in order to induce tolerance in a subject. The disclosed methods may be performed in order to reduce and/or eliminate a T cell response and/or to induce tolerance to an antigen (e.g., an autoantigen). In some embodiments, the disclosed methods may be performed in order treat and/or prevent an autoimmune disease or disorder in a subject in need thereof.

The cells disclosed herein typically are genetically modified cells comprising an inserted exogenous sequence in a major histocompatibility complex (MHC)-associated gene. As disclosed herein, the term “exogenous” refers to a polynucleotide sequence that is not present in the non-modified MHC-associated gene. An “exogenous” polynucleotide sequence may refer to a polynucleotide sequence occurring elsewhere in a modified cell other than in the MHC-associated gene. An “exogenous” sequence also may refer to a polynucleotide sequence that is not present in the modified cell, such as a polynucleotide sequence that is present in a different cell-type than the cell-type of the modified cell. An “exogenous” sequence may refer to a polynucleotide sequence that is present in a different organism than the organism from which the modified cell is derived (e.g., a microbial organism, a fungal organism, or a virus). An “exogenous” sequence also may refer to a polynucleotide sequence that is artificial and is not observed to occur naturally in any organism.

The inserted polynucleotide sequence typically encodes a peptide which may be an antigen or which functions as an antigen. Antigens encoded by the polynucleotide sequence may include foreign antigens or heteroantigens which may be defined as antigens that are not present and/or expressed in the organism from which the genetically modified cells are derived. In some embodiments, the disclosed modified cells may express a fusion protein comprising a foreign antigen or heteroantigen derived from a microorganism (e.g., a virus, bacteria, or fungus).

Antigens encoded by the inserted polynucleotide sequence may include antigens that are associated with a cancer and may be referred to as neoantigens or tumor-specific antigens. Neoantigens may be defined as antigens comprising non-synonymous mutations relative to the non-mutant containing gene from which the neoantigens are derived. Neoantigens typically are not expressed in normal tissues and are highly immunogenic. In some embodiments, the disclosed modified cells may express a fusion protein comprising a neoantigen.

Antigens encoded by the inserted polynucleotide sequence may include autoantigens or self-antigens which are present and expressed in the organism from which the modified cells are derived. In some embodiments, the disclosed modified cells may express a fusion protein comprising an antigen expressed in the organism from which the modified cells are derived.

The inserted exogenous sequence encodes a peptide and optionally may include a linking amino acid sequence that links the encoded peptide to the amino acid sequence of the polypeptide encoded by the MHC-associated gene. Linker sequences for fusion proteins have been described. (See Chen, Xiaoying et al. “Fusion protein linkers: property, design and functionality.” Advanced drug delivery reviews vol. 65,10 (2013): 1357-69, the content of which is incorporated by reference in its entirety). In some embodiments, the linker comprises 5, 6, 7, 8, 9, 10, 15, 20, 25, or 30 amino acids. The linker may be relatively flexible. In some embodiments, the linker comprises or consists of amino acid sequence selected from glycine (G), serine (S), and alanine (A). Optionally, the linker has a sequence (G₄S)ₙ where n is 3-6 or Gₙ where n is 3-10. The linker may be relatively rigid. In some embodiments, the linker may have a sequence (EAAAK (SEQ ID NO: 3))ₙ where n is 3-6 or (XP)ₙ where n is 3-10. The inserted exogenous sequence may encode a linker fused to the N-terminus of the peptide, a linker fused to the C-terminus of the peptide, or linkers fused to both of the N-terminus and C-terminus of the peptide.

The genetically modified cells disclosed herein typically express a fusion protein that comprises an exogenous peptide fused directly or indirectly via a linker to at least a portion of a polypeptide encoded by an MHC-associated gene. An MHC associated gene is any gene encoding an MHC protein and/or any gene encoding proteins that associate with MHC proteins or the MHC complex (e.g., B2M and chaperone proteins such as CD74).

In some embodiments, the genetically modified cells express a fusion protein comprising a peptide fused to at least a portion of a polypeptide encoded by a MHC class I associated gene, which may include human leukocyte antigen A (HLA-A), HLA-B, HLA-C, HLA-E, HLA-G, HLA-H, HLA-J, HLA-K, and HLA-L. In some embodiments, the genetically modified cells express a fusion protein comprising a peptide fused to at least a portion of beta-2 microglobulin (B2M).

The inserted exogenous sequence encodes a peptide which may be presented as an antigen. In some embodiments, the exogenous sequence encodes a peptide and optionally a linker. In this embodiment, the exogenous sequence may be inserted into an MHC-associated gene in-frame in order to create a fusion protein comprising the peptide fused directly or indirectly via the optional linker to at least a portion of a polypeptide encoded by the MHC-associated gene to form a modified MHC complex such that the peptide is presented at the peptide binding cleft of the modified MHC complex.

In other embodiments, the exogenous sequence encodes a fusion protein comprising a peptide fused directly or indirectly via an optional linker to at least a portion of a polypeptide encoded by an MHC-associated gene. In this embodiment, the exogenous sequence may be inserted into an endogenous MHC-associated gene in order to knock-in the exogenous sequence and knock-out the endogenous MHC-associated gene. In the context of the fusion protein expressed by the genetically modified cell, the peptide portion of the fusion protein may be presented at the peptide binding cleft of the protein encoded by the MHC-associated gene.

The exogenous sequence encoding the peptide may be inserted at any suitable location of the MHC-associated gene so that the peptide is presented at the peptide binding cleft of the modified MHC complex. In some embodiments, the exogenous sequence is inserted at the 5′ region of the MHC-associated gene to create a fusion protein comprising the peptide fused either directly or via a linker to the N-terminus of at least a portion of the polypeptide encoded by the MHC-associated gene. The fusion protein may have a sequence represented as: N-(peptide)-(optional linker)-(at least a portion of the polypeptide encoded by the MHC-associated gene)-C.

In other embodiments, the exogenous sequence encoding a peptide is inserted at the 3′ region of the MHC-associated gene to create a fusion protein comprising the peptide fused either directly or via a linker to the C-terminus of at least a portion of the polypeptide encoded by the MHC-associated gene such that the peptide is presented at the peptide binding cleft of the modified MHC complex. The fusion protein may have a sequence represented as: N-(at least a portion of the polypeptide encoded by the MHC-associated gene)-(optional linker)-(peptide)-C.

In even further embodiments, the exogenous sequence encoding a peptide is inserted internally within the MHC-associated gene to create a fusion protein comprising an N-terminal portion of the polypeptide encoded by the MHC-associated gene fused either directly or indirectly via an optional linker to the peptide, which in turn is fused either directly or indirectly via an optional linker to a C-terminal portion of the polypeptide encoded by the MHC-associated gene, such that the peptide is presented at the peptide binding cleft of the modified MHC complex. The fusion protein may have a sequence represented as: N-(at least a portion of the polypeptide encoded by the MHC-associated gene)-(optional linker)-(peptide)-(optional linker)-(at least a portion of the polypeptide encoded by the MHC-associated gene-C.

The fusion proteins that are expressed by the genetically modified cells typically include at least a portion of a polypeptide encoded by an MHC-associated gene. In some embodiments, the fusion proteins comprise two or more portions from two or more polypeptides encoded by MHC-associated genes which may be contiguous or non-contiguous. In some embodiments, the fusion proteins comprise, from N-terminus to C-terminus, a peptide fused via a linker to a B2M polypeptide, which in turn is fused via a linker to an HLA polypeptide (i.e., represented as N-peptide-linker-B2M-linker-HLA-C). In some embodiments, the fusion proteins expressed by the genetically modified cells comprise a signal peptide of a polypeptide encoded by an MHC-associated gene. For example, the fusion proteins may comprise the signaling peptide (SP) of a B2M polypeptide (i.e., represented as N-SP-peptide-linker-B2M-linker-HLA-C). The signaling peptide may be cleaved and the fusion protein may be expressed on the cell surface of the genetically modified cell.

Suitable insertion sites for the exogenous sequence encoding the peptide and optionally the linker may include a site between a signaling peptide and a mature protein encoded by an MHC-associated gene. For example, the exogenous sequence may be inserted to provide a fusion protein having a sequence N-(signaling peptide)-(exogenous peptide)-(mature protein)-C.

Suitable insertion sites for the exogenous sequence encoding the peptide and optionally the linker may include a site encoding the peptide binding cleft of the class I MHC complex.

Suitable insertion sites for the exogenous sequence encoding the peptide and optionally the linker may also include a site encoding the N-terminus of the chain of a class I MHC complex, N-terminus of B2M and/or C-terminus of B2M such that the peptide is presented at the peptide binding cleft of the modified MHC complex.

Suitable regions for inserting the exogenous sequence and preparing a fusion protein may be selected via performing an analysis of conserved regions within a polypeptide encoded by an MHC-associated gene, such as HLA-A, HLA-B, and HLA-C. Conserved regions within MHC-associated proteins are well known see, e.g., http://hla.alleles.org/alleles/heat_maps.html for heat maps of HLA-A, HLA-B and HLA-C illustrating conserved regions. In some embodiments, the exogenous sequence encoded the peptide is inserted in-frame within HLA-A at amino acids sequence from: aa 1-61, aa 117-152, aa 167-182, or aa 215-274. In some embodiments, the exogenous sequence encoded the peptide is inserted in-frame within HLA-B at amino acids sequence from: 47-62, aa 117-160, or aa 182-273. In some embodiments, the exogenous sequence encoded the peptide is inserted in-frame within HLA-C at amino acids sequence from: aa 25-72, aa 117-145, or aa 164-283.

In some embodiments, the disclosed genetically modified cells are prepared from antigen presenting cells. Suitable antigen presenting cells may include cells such as dendritic cells, macrophages, monocytes, or a B cell.

The genetically modified cells preferably express the fusion proteins on their cell surface in a manner whereby the fusion proteins can be recognized by immune cells such as T cells. In some embodiments, the fusion proteins are expressed in a manner which mimics a native MHC complex (i.e., the fusion proteins form at least part of a modified MHC complex comprising the fusion proteins) and in a manner whereby the exogenous peptide is expressed in the peptide binding cleft and is presented to T cells. Preferably, T cells can bind to the genetically modified cells via an interaction between the T cell receptor and the modified MHC complex comprising the fusion protein. Preferably, the genetically modified cells activate T cells, for example, via an interaction between the T cell receptor and the modified MHC complex comprising the fusion protein of the genetically modified cells. Preferably, T cells are activated against the exogenous peptide of the fusion protein.

The genetically modified cells may express an antigen-containing fusion protein, for example, as part a modified MHC complex. Thus, the genetically modified cells may be utilized in methods for modulating an immune response in vitro or in vivo.

In some embodiments, the genetically modified cells may be utilized in methods for activating T cells in vitro or in vivo. The disclosed methods may include contacting the T cells with the genetically modified cells under conditions whereby the T cells are activated. In some embodiments of the disclosed methods, the T cells are activated in vitro. For example, T cells may be explanted from a donor, activated in vitro, and optionally, transplanted back to the donor or to another recipient. In other embodiments of the disclosed methods, the genetically modified cells may be administered to a subject in vivo in order to activate T cells within the subject.

In some embodiments, the genetically modified cells may be utilized in methods for inducing T cell tolerance. The disclosed methods may include contacting the T cells with the genetically modified cells under conditions whereby tolerance is induced in the T cells. In some embodiments, the genetically modified cells may be administered to a subject in vivo in order to induce tolerance in T cells of the subject.

The genetically modified cells may be administered to a subject, for example, as part of a pharmaceutical compositions comprising the genetically modified cells and a suitable pharmaceutical carrier. The genetically modified cells may be administered to a subject in order to modulate an immune response in the subject. In some embodiments, the genetically modified cells are administered to a subject in order to activate an immune response against an antigen in the subject (e.g., a foreign antigen or neoantigen expressed by the genetically modified cells as a fusion protein). In other embodiments, the genetically modified cells are administered to a subject in order to induce tolerance to an antigen in the subject (e.g., an autoantigen expressed by the genetically modified cells as a fusion protein).

The genetically modified cells may be administered to a subject in order to treat and/or prevent a disease or disorder in the subject. In some embodiments, the genetically modified cells may be administered to a subject in order to prevent the occurrence or recurrence of a disease or disorder in the subject.

T cells that have been activated by the disclosed genetically modified cells also may be administered to a subject in order to treat and/or prevent a disease or disorder in the subject. In some embodiments, T cells that have been activated by the disclosed genetically modified cells may be administered to a subject in order to prevent the occurrence or recurrence of a disease or disorder in the subject.

The genetically modified cells and/or T cells that are administered to the subject in the disclosed methods may be derived from the subject and/or may be derived from another donor. As such, suitable cells for performing the disclosed methods may be autologous or allogeneic relative to a subject who donated the cells and/or relative to a subject who is a recipient of the cells. For example, a subject may donate a cell which is genetically modified as disclosed herein (e.g., ex vivo), and the genetically modified cell then may be administered to the subject in a method of treatment. In another embodiment, a cell may be obtained from a donor subject who is allogeneic relative to a recipient subject to which the cell will be administered after the cell has been genetically modified (e.g., ex vivo) as disclosed herein.

Diseases and disorders that may be treated and/or prevented by the disclosed methods may include, but are not limited to, proliferative cell diseases and disorders such as cancers. In some embodiments of the disclosed methods, a subject is administered genetically modified cells that express a fusion protein comprising a neoantigen or tumor specific antigen or a subject is administered T cells that have been activated by genetically modified cells that express a fusion protein comprising a neoantigen or tumor specific antigen.

Disease and disorders that may be treated and/or prevented by the disclosed methods may include, but are not limited to, infectious diseases (e.g., viral infections, bacterial infections, fungal infections, and the like). In some embodiments of the disclosed methods, a subject is administered genetically modified cells that express a fusion protein comprising an antigen of an infectious agent or a subject is administered T cells that have been activated by genetically modified cells that express a fusion protein comprising an antigen of an infectious agent.

Diseases and disorders that may be treated and/or prevented by the disclosed methods may include, but are not limited to, autoimmune diseases (e.g., type 1 diabetes, multiple sclerosis, lupus, and rheumatoid arthritis). In some embodiments of the disclosed methods, a subject is administered genetically modified cells that express a fusion protein comprising an autoantigen or a subject is administered T cells that have been contacted by genetically modified cells that express a fusion protein comprising an autoantigen to induce tolerance to the autoantigen.

The genetically modified cells may be prepared using recombination methods known in the art. In some embodiments, the genetically modified cells are prepared using homologous recombination methods (e.g., microhomology-mediated end joining or homology directed repair). In other embodiments, the genetically modified cells are prepared using non-homologous recombination methods (non-homologous end joining).

The genetically modified cells may be prepared by recombination methods that utilize nucleases to promote recombination at selected genomic sites (e.g., as effector proteins). Suitable nucleases may include clustered repeat interspaced short palindromic repeats (CRISPR) effector polypeptides, for example, a type II CRISPR effector polypeptide such as a Cas9 polypeptide and type V CRISPR effector polypeptides such as a Cas12a, a Cas12b, a Cas12c, a Cas12d, a Cas12e, a Cas12f, a Cas12g, a Cas12h or a Cas12i polypeptide). Suitable CRISPR-effector polypeptides also may include a Cas14a, a Cas14b, or a Cas14c polypeptide.

Suitable nucleases for preparing the genetically modified cells may include non-CRISPR effector polypeptides. Other suitable nucleases may include zinc finger nucleases (ZFNs) and transcription activator-like effector nucleases (TALENs).

In some embodiments, the genetically modified cells may be prepared by a method comprising introducing into a cell: (a) a CRISPR effector protein or a polynucleotide encoding a CRISPR effector protein; (b) a guide polynucleotide comprising a guide sequence designed to hybridize with a target sequence in the MHC-associated gene in the cell; and (c) a donor polynucleotide comprising a polynucleotide sequence encoding the peptide (e.g., an exogenous sequence). In the disclosed methods, preferably the CRISPR effector protein introduces a double-stranded break at the target sequence and repair of the double-stranded break through a DNA repair process results in insertion of the inserted polynucleotide sequence encoding the peptide in the MHC-associated gene in the cell thereby producing a modified cell expressing a genetically modified MHC-associated gene. In some embodiments, the inserted polynucleotide sequence encoding the peptide may be inserted in-frame with the coding sequence of the MHC-associated gene such that the genetically modified MHC-associated gene encodes a novel fusion protein. In other embodiments, the inserted polynucleotide sequence encodes a fusion protein comprising the peptide fused to a polypeptide encoded by an MHC-associated gene, and the insertion knocks out an endogenous MHC-associated gene.

The disclosed methods for preparing the genetically modified cells may be performed ex vivo or in vivo. For example, the disclosed methods for preparing the genetically modified cells may be performed in vivo in a subject in order to create genetically modified cells in the subject and treat and/or prevent a disease or disorder in the subject as disclosed herein. In such methods, the subject may be administered: (a) a CRISPR effector protein or a polynucleotide encoding a CRISPR effector protein; (b) a guide polynucleotide comprising a guide sequence designed to hybridize with a target sequence in the MHC-associated gene in a cell of the subject; and (c) a donor polynucleotide comprising a polynucleotide sequence encoding the peptide (e.g., an exogenous sequence). In the disclosed methods, preferably the CRISPR effector protein introduces a double-stranded break at a target sequence of a target cell and repair of the double-stranded break through a DNA repair process results in insertion of the inserted polynucleotide sequence encoding the peptide in the MHC-associated gene in the target cell thereby producing a modified cell expressing a genetically modified MHC-associated gene in the subject. In some embodiments, the inserted polynucleotide sequence encoding the peptide may be inserted in-frame with the coding sequence of the MHC-associated gene such that the genetically modified MHC-associated gene encodes a novel fusion protein. In other embodiments, the inserted polynucleotide sequence encodes a fusion protein comprising the peptide fused to a polypeptide encoded by an MHC-associated gene, and the insertion knocks out an endogenous MHC-associated gene.

As indicated, patient or donor antigen presenting cells (APC) can be edited ex vivo or in vivo. In the ex vivo setting immune cells can be isolated and edited in bulk or following separation into immune cell subsets such as T cells, stem cells, and APC. Separation technologies can include flow cytometry, antibody bead-based separation, aptamers or other physical methods for separation. Gene editing can be performed using guide RNA and a suitable genome editing enzyme and can be delivered using viral or non-viral gene delivery methods. Following editing, patient APC can be returned for benefit. Patient APC can also be gene edited in vivo, whereby viral or non-viral delivery methods would target APC in circulation or in situ. This might also be accomplished using catheter-based delivery of desired editing complex. This can include the ability to control gene editing in vivo or ex vivo using alternate guide chemistry or protein design. This can include other biophysical and interventional techniques used to deliver gene, cell or biologics therapies in vivo.

In the disclosed methods for preparing genetically modified cells, suitable donor polynucleotides may include single stranded DNA and/or double stranded DNA. Vectors may be utilized in order to provide donor polynucleotides in the disclosed methods. Suitable vectors may include viral vectors, plasmids, and transposons.

Also disclosed herein are systems and kits comprising the disclosed genetically modified cells and configured for preforming the disclosed methods. The systems and kits may comprise and/or utilize the genetically modified cells, T cells whose activity has been modified by the genetically modified cells, and devices or instructions for using the system and kits.

### EXAMPLES

The following examples are illustrative and should not be interpreted to limit the scope of the claimed subject matter.

### Example 1

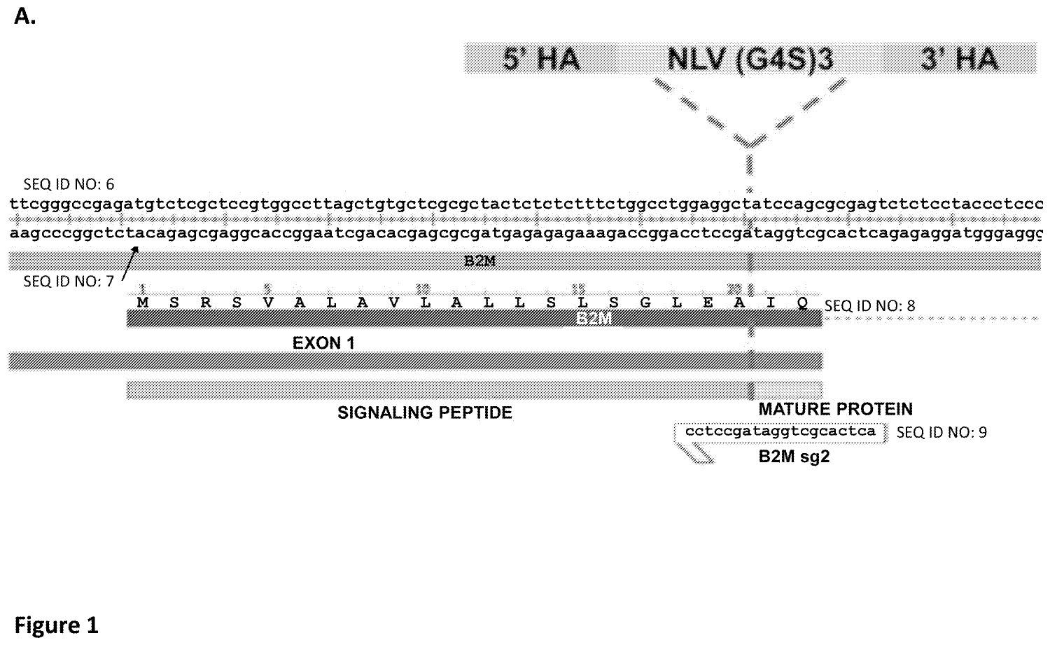

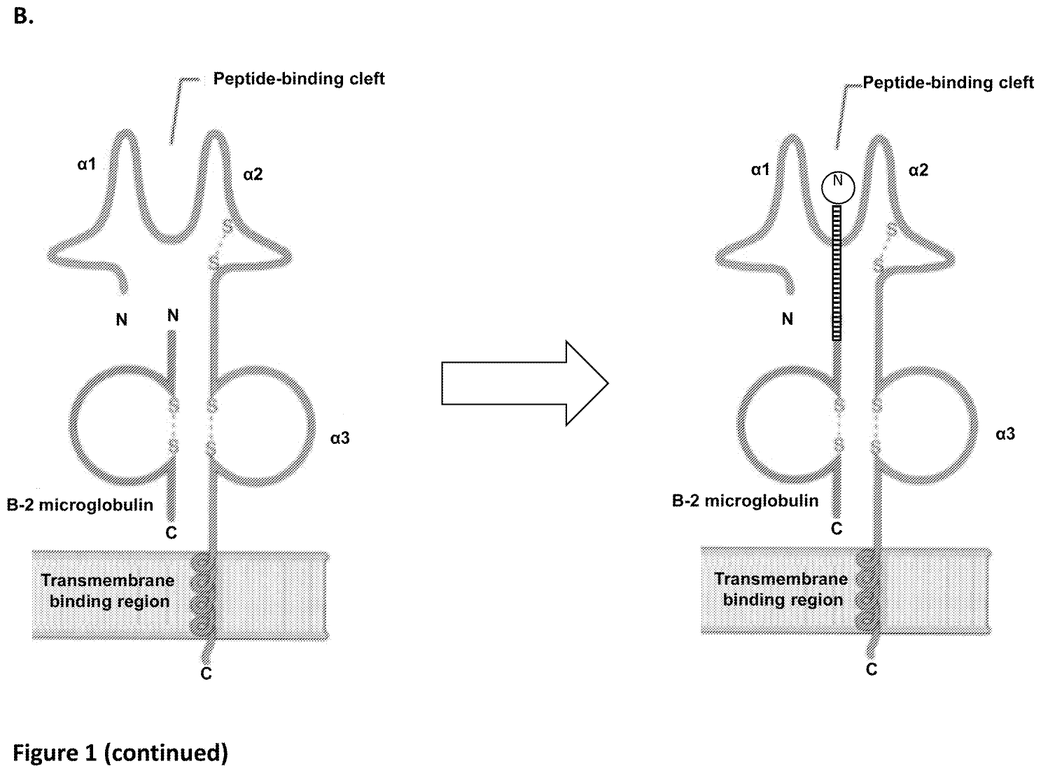

Editing of MHC-associated genes and insertion of an exogenous sequence encoding an antigenic peptide. An exogenous sequence encoding a peptide NLVPMVATV (SEQ ID NO: 1) (“NLV peptide”) and a linker was knocked-in between the beta-2 microglobulin (B2M) signaling peptide and the mature B2M protein. (See FIG. 1A). As such, the coding sequence for a novel fusion protein having a sequence N-peptide-linker-B2M was created. As indicated, the NLV peptide is fused to the N-terminus of B2M by a (G₄S)₃ linker and is presented in the peptide-binding cleft of the MHC complex. (See FIG. 1B)

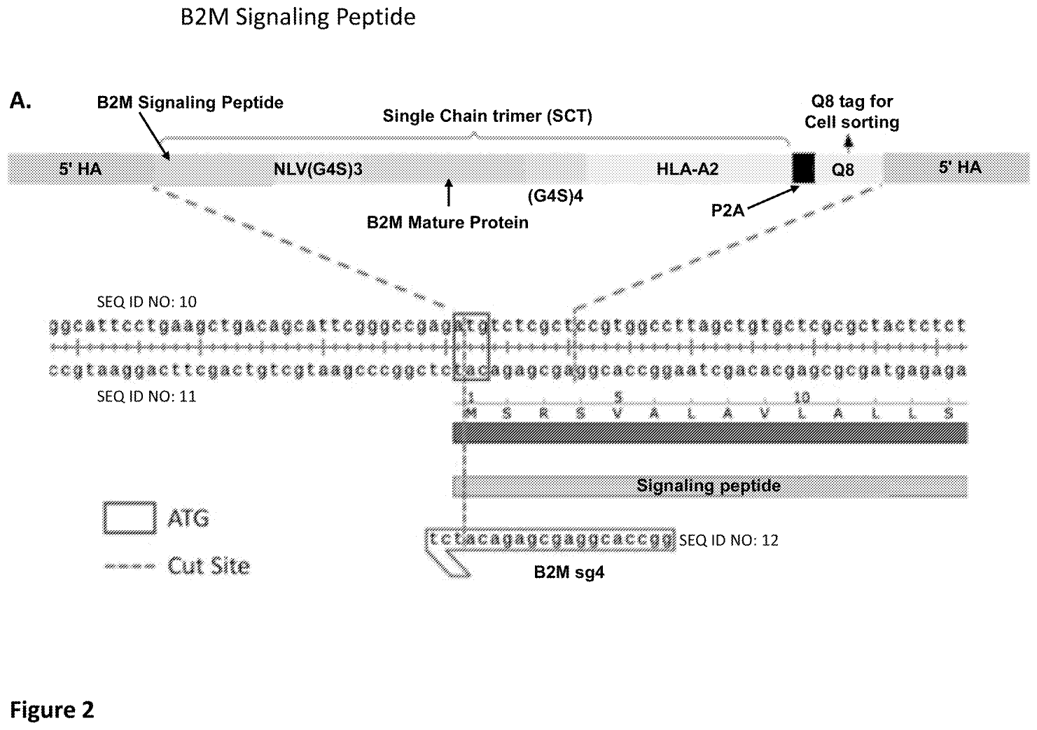

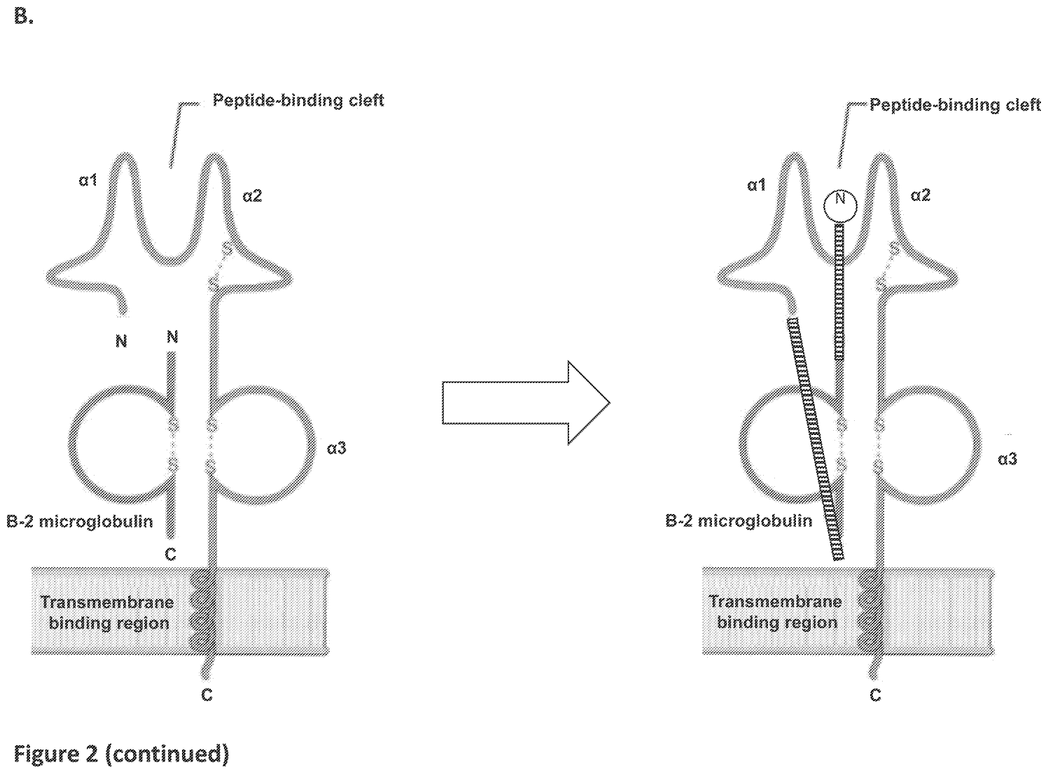

Insertion of exogenous sequence encoding a fusion protein, and replacement of endogenous MHC-associated genes. An exogenous sequence encoding a fusion protein comprising a NLV peptide single chain trimer (SCT) was knocked-in at the 5′ end of the B2M coding region. (See FIG. 2A). The SCT trimer comprises: the B2M signaling peptide fused to the encoded NLV peptide; which in turn is fused to the N terminus of B2M via a (G₄S)₃ linker; which in turn is fused to the N-terminus of the HLA-A2 polypeptide via a (G₄S)₄ linker. The fusion protein further includes a C-terminal P2A sequence fused to the C-terminus of the HLA-A2 polypeptide and a Q8 tag. The SCT was knocked-in in a manner which abolishes expression of the native MHC-associated genes. The SCT displays the encoded NLV peptide in the binding cleft of the modified MHC complex. (See FIG. 2B).

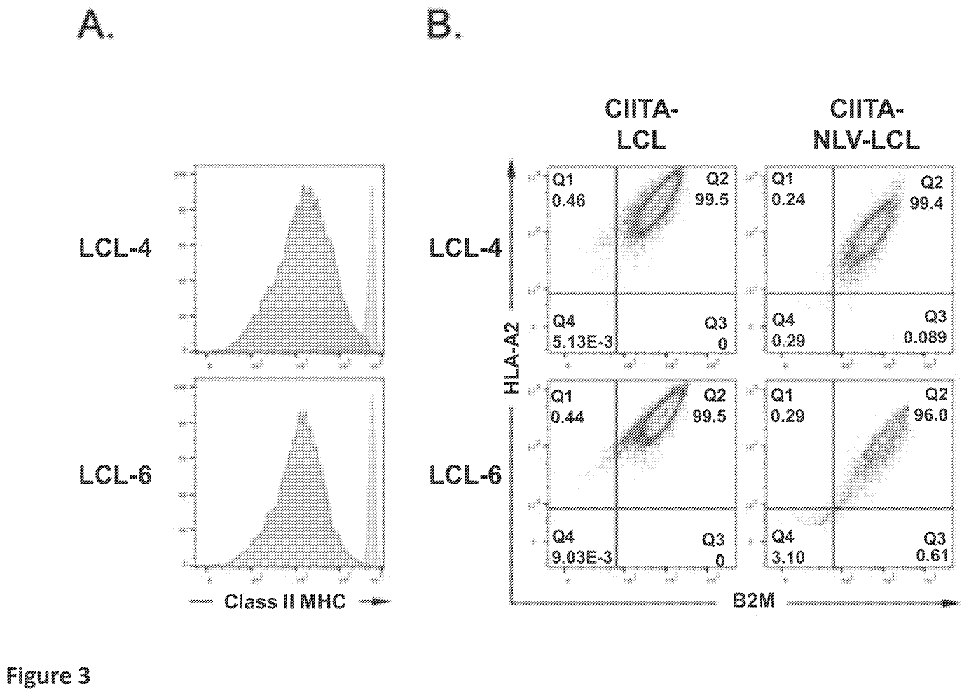

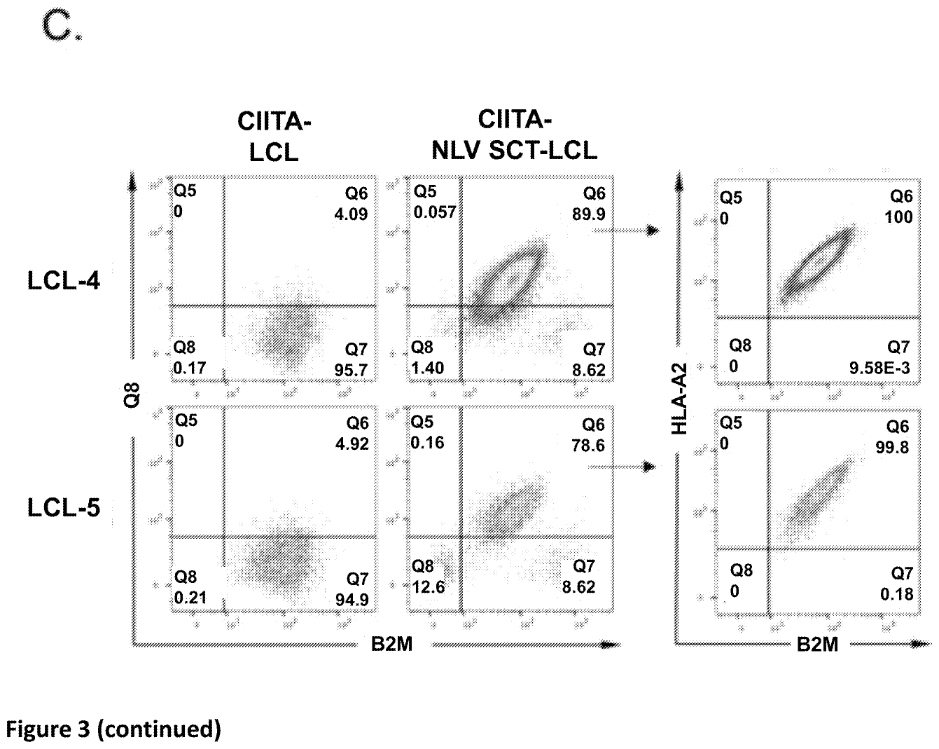

Engineering of lymphoblastoid cell lines (LCLs) to express NLV peptide and FACS analysis. The CIITA (class II transactivator) gene was knocked-out from three different HLA-A2+ LCL lines (i.e., LCL-4, LCL-5, and LCL6). FACS analysis then was performed to assess expression of CIITA. (See FIG. 3A). NLV peptide then was knocked-in to selected lines. FACS analysis of HLA-A2 and B2M expression in CIITA⁻ LCL lines and NLV peptide knock-in LCL lines then was performed. (See FIG. 3B). FACS analysis of Q8, B2M and HLA-A2 expression in CIITA⁻ LCL lines and SCT knock-in LCL lines also was performed. (See FIG. 3C).

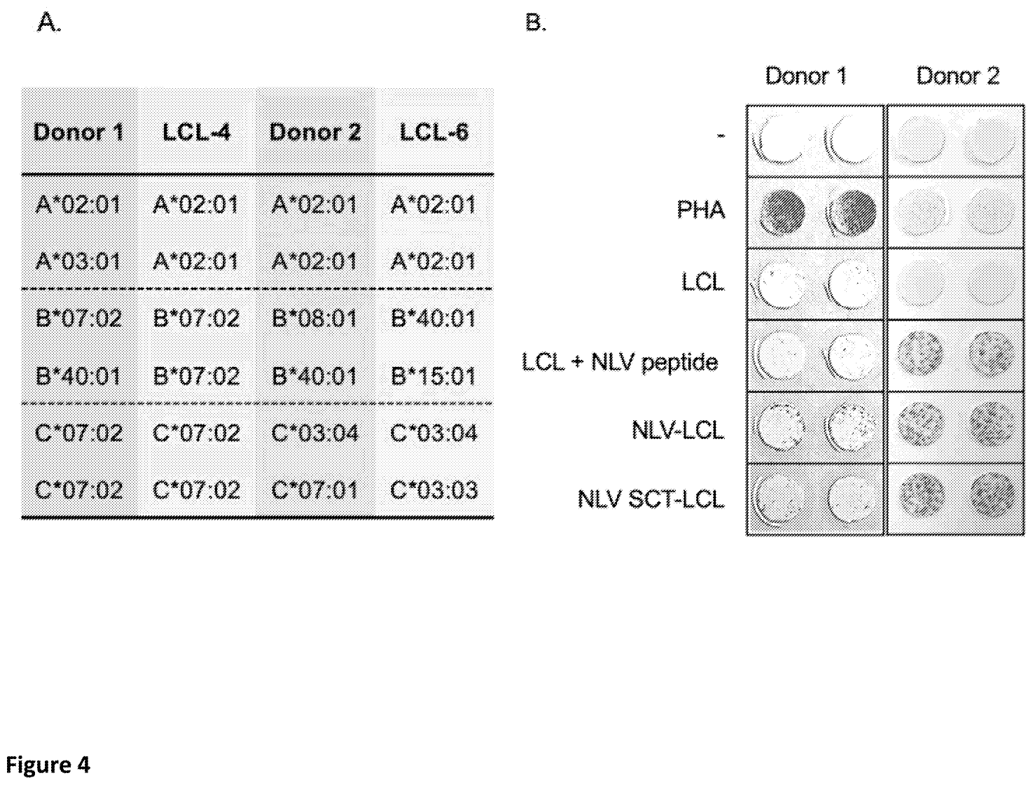

T cell activation by edited antigen presenting cells (APCs). The HLA-types of two healthy donors (i.e., Donor 1 and Donor 2) and two edited LCL lines (i.e., LCL-4 and LCL-6) were compared and matched. (See FIG. 4A). The class I HLA of Donor 1 and Donor 2 were matched with LCL-4 and LCL-6, respectively. The edited LCL-4 and LCL-6 lines, which express NLV-peptide, were used as antigen presenting cells (APCs) for the NLV-specific CTL lines, and IFNγ ELISPOT was used to assess activation. (See FIG. 4B). The results demonstrate that the NLV peptide could be directly knocked-in to the N-terminus of mature B2M gene and presented by the class I MHC to activate T cells. For Donor 1, the edited LCL line provided stronger stimulation than peptide pulsed APCs or single chain trimer MHC complex-expressing APCs.

### Materials and Methods

Primary cells and cell lines. CMV seropositive frozen PBMCs were purchased from PPA/BioIVT. Class I HLA matched LCL lines were purchased from International histocompatibility working group, Fred Hutch.

Engineering of APC. Two B2M guide RNAs (B2M sg2: ACTCACGCTGGATAGCCTCC (SEQ ID NO: 4); B2M sg4: GGCCACGGAGCGAGACATCT (SEQ ID NO: 5)) were synthesized by Synthego. Single strand DNA for knock-in NLV peptide with linker into B2M locus, and plasmid for knock-in NLV-single chain trimer were synthesized by Genwiz. LCLs may be edited via the Jiang et al. (“CRISPR/Cas9-Mediated Genome Editing in Epstein-Barr Virus-Transformed Lymphoblastoid B-Cell Lines,” Current protocols in molecular biology vol. 121 31.12.1-31.12.23. 16 Jan. 2018). Following electroporation, LCLs were recovered and expanded in RPMI supplemented with 15% FBS and sodium pyruvate.

Flow cytometry. Viability dye zombie NIR, anti-B2M, HLA-A2 and HLA-DR/DP/DQ were purchased from biolegend; anti-CD34 (Clone Qbent 10) were purchased from Thermofisher. Cells were stained with antibody cocktails in FACS staining buffer or culture medium for 15 min at 4 degree. All flow cytometry data were obtained in MACSquant analyzer (Miltenyi) and analyzed with Flowjo software (Flowjo).

ELISPOT. 96-well MultiScreen HTS IP plates (EMD Millipore, MA) coated with anti-human IFNγ mAb 1-DIK (Mabtech), and kept overnight at 4° C. NLV-specific T cells (2k for donor 1 and 50k for donor 2, in duplicate) were plated in duplicate, and stimulated CIITA negative LCL pulsed with NLVPMVATV (SEQ ID NO: 1) peptide (JPT Technology), NLV peptide expressing-CIITA negative LCL line, NLV single chain trimer-expressing CIITA negative LCL, and with PHA (eBioscience) as a positive control. After 16-20 hours at 37° C., the cells were completed removed, and 96-well plate was incubated with anti-human IFNγ mAb 7-B6-1-biotin (Mabtech) for 2 hours at 37° C., and avidin-peroxidase-complex (Vector Laboratories) was added for 1 hour at room temperature. The plates were then developed with 3-amino-9-ethylcarbazole (Sigma) substrate, dried.

### Example 2

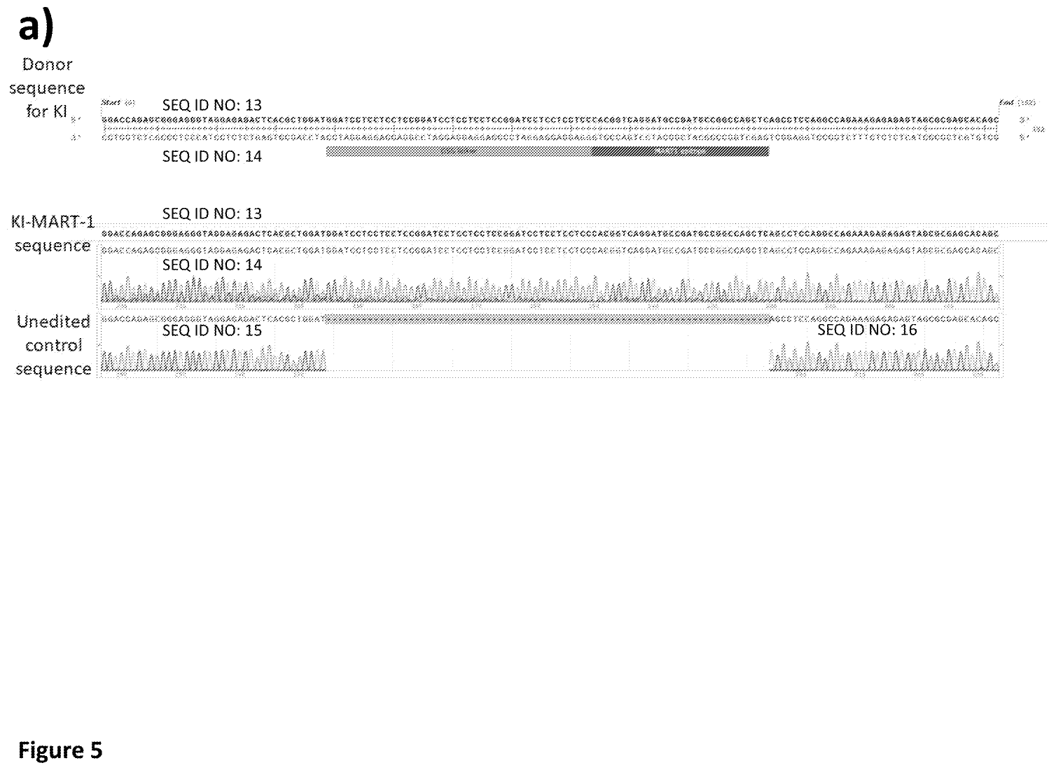

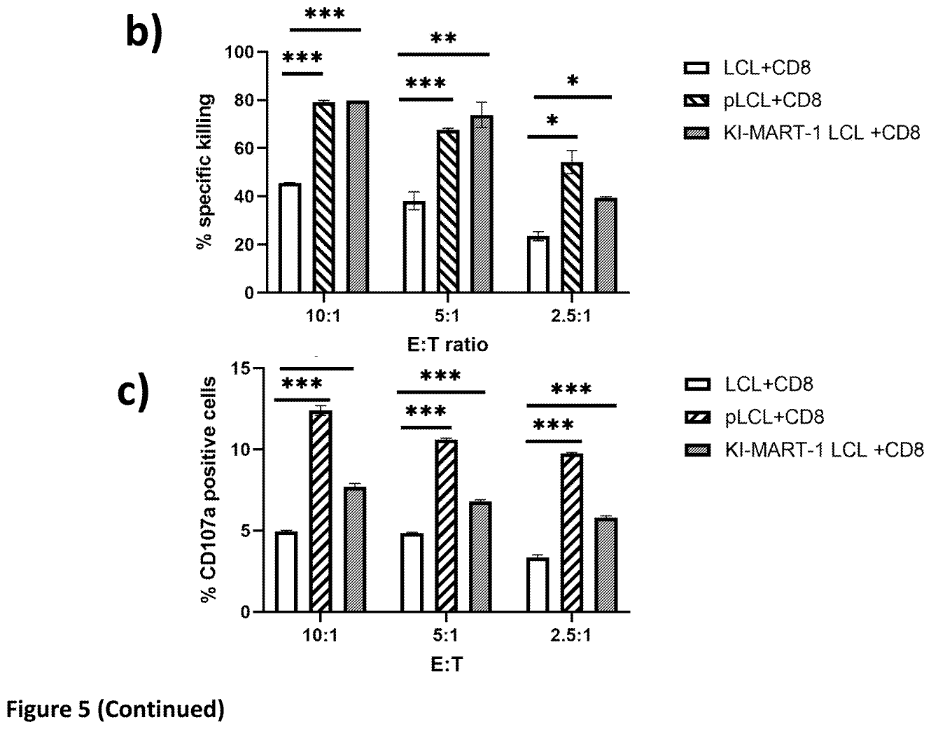

Insertion of antigen peptide elicits antigen-specific CD8+ T cell responses. Lymphoblast cell line cells (LCLs) were engineered to express the melanoma antigen MART-1 peptide (ELAGIGILTV(SEQ ID NO: 2)) tethered to the beta-2 microglobulin (B2M) subunit of HLA class I. (See FIG. 5a). Edited cells were then co-cultured overnight with commercially acquired donor-derived CD8+ cytotoxic T cells specifically reactive to the ELAGIGILTV(SEQ ID NO: 2) peptide (HemaCare Cellero) at defined effector:target (E:T) ratios. (See FIG. 5b). Particularly, MART-1 specific CD8+ T cells were mixed at three different E:T ratios with pre-stained HLA-matched LCL either pulsed with MART-1 peptide (pLCL) or engineered to express the MART-1 epitope in the context of HLA-A*0201 (KI MART-1LCL). Cells were co-cultured overnight before flow cytometry analysis. The percentage of surface-exposed CD107a (LAMP1) positive CD8+ T cells was assayed. (See FIG. 5c). A higher percentage of surface-exposed CD107a (LAMP1) positive CD8+ T cells was observed to correlated with CD8+ T cell activation, target-specific recognition, and killing.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

1. A cell comprising a genetically modified major histocompatibility complex (MHC) associated gene, wherein the genetically modified MHC-associated gene has been genetically modified to comprise an inserted polynucleotide sequence encoding a peptide and optionally a linker such that the genetically modified MHC-associated gene encodes a fusion protein comprising the peptide and optional linker.

2. The cell of claim 1, wherein the peptide comprises a non-self antigen, a self-antigen, or a neoantigen.

3. (canceled)

4. (canceled)

5. The cell of claim 1, wherein the linker comprises 5, 6, 7, 8, 9, 10, 15, 20, 25, or 30 amino acids select from G, S, and A, and optionally comprises (G₄S)ₙ where n is selected from 3-6.

6. (canceled)

7. The cell of claim 1, wherein the cell is capable of presenting the peptide via the MHC to a T cell to induce activation or tolerance and/or the cell presents the peptide via the MHC to a T cell to induce activation or tolerance.

8. The cell of claim 1, wherein the inserted polynucleotide sequence encodes a fusion protein comprising the peptide and optionally a linker fused in-frame to at least a portion of a coding sequence of an MHC-associated gene and the inserted polynucleotide sequence knocks-out an endogenous MHC-associated gene.

9. The cell of claim 8, wherein the endogenous MHC-associated gene is a B2M gene.

10. The cell of claim 1, wherein the inserted polynucleotide sequence does not comprise an MHC-associated gene and the peptide and the optional linker are inserted in-frame with a coding sequence of an endogenous MHC-associated gene.

11. The cell of claim 1, wherein the peptide does not comprise a signal peptide encoded by an MHC-associated gene.

12. The cell of claim 1, wherein the B2M gene in the cell is not knocked out.

13. The cell of claim 1, wherein the inserted polynucleotide sequence produces a homologous knock-in in the cell.

14. The cell claim 1, wherein the MHC-associated gene is the B2M gene.

15. The cell of claim 14, wherein the inserted polynucleotide sequence is inserted in a region of the B2M gene that encodes for the N-terminus of the B2M protein or the C-terminus of the B2M protein.

16. The cell of claim 14, wherein the inserted polynucleotide sequence is inserted in a region of the B2M gene that encodes for the first twenty amino acids encoding region of the B2M protein.

17. The cell of claim 1, wherein the genetically modified MHC-associated gene has been further genetically modified to comprise a second inserted polynucleotide sequence encoding a second MHC-associated gene and optionally a linker such that the genetically modified MHC-associated gene encodes a fusion protein comprising the peptide and optionally the linker.

18. The cell of claim 17, wherein the second inserted polynucleotide sequence is inserted in a region that encodes for the B2M protein.

19. The cell of claim 17, wherein the second inserted polynucleotide sequence is inserted in a region of the B2M gene that encodes for the C-terminus of the B2M protein.

20. The cell of claim 1, wherein the MHC-associated gene is an MHC class I gene.

21. The cell of claim 20, wherein the MHC class I gene is an HLA class I gene.

22. The cell of claim 21, wherein the HLA class I gene is a gene encoding an HLA-A protein, an HLA-B protein, an HLA-C protein, an HLA-E protein, an HLA-L protein, an HLA-J protein, an HLA-K protein, an HLA-H protein, or an HLA-G protein.

23. (canceled)

24. The cell of claim 1, wherein the MHC-associated gene is a B2M gene encoding a B2M protein having a N-terminus and a C-terminus, wherein the inserted polynucleotide sequence encoding the peptide is inserted in frame with the N-terminus of the B2M protein and optionally further comprising a second inserted polynucleotide encoding a second peptide linked in frame to the C-terminus of the B2M protein and wherein the second polynucleotide encoding the second peptide is further optionally linked in frame to a polynucleotide encoding a MHC class I protein.

25. The cell of claim 1, wherein the cell is an antigen presenting cell (APC) or an artificial antigen presenting cell (aAPC).

26. The cell of claim 25, wherein the APC is a dendritic cell, a macrophage, a monocyte or a B cell.

27. (canceled)

28. (canceled)

29. The cell of claim 1, wherein the cell is an autologous or allogeneic cell relative to a recipient subject to which the cell may be administered.

30. (canceled)

31. A method of activating a T cell comprising contacting the T cell with the cell of claim 1.

32. A method of inducing tolerance in a T cell, the method comprising contacting the T cell with the cell of claim 1.

33. (canceled)

34. A method of the cell of claim 1, the method comprising introducing into the cell:
a. a CRISPR effector protein or a polynucleotide encoding a CRISPR effector protein;
b. a guide polynucleotide comprising a guide sequence designed to hybridize with a target sequence in the MHC-associated gene in the cell; and
c. a donor polynucleotide comprising a polynucleotide sequence encoding the peptide;
wherein the CRISPR effector protein introduces a double-stranded break at the target sequence and repair of the double-stranded break through a DNA repair process results in insertion of the inserted polynucleotide sequence encoding the peptide in the MHC-associated gene in the cell thereby producing a modified cell expressing a genetically modified MHC-associated gene, wherein the genetically modified MHC-associated gene encodes a fusion protein comprising the peptide.

35. (canceled)

36. A method of modulating an immune response in a subject comprising administering the cell of claim 1 to the subject.

37. A method of modulating an immune response in a subject comprising administering a population of activated or tolerogenic T cells to the subject, wherein the population of activated or tolerogenic T cells are produced by contacting the cell of claim 1 with a T cell or a population of T cells.

38.-47. (canceled)

48. The method of claim 34, wherein the CRISPR effector protein is a type II or type V CRISPR effector protein.

49.-51. (canceled)

52. The method of claim 34, wherein the CRISPR effector protein is Cas14a, a Cas14b, or a Cas14c polypeptide.

53.-62. (canceled)

63. The method of claim 31, wherein the T cell is an effector T cell, a cytotoxic T cell, a helper T cell, or a regulatory T cell.

64.-87. (canceled)

88. The method of claim 36, wherein the subject is a human.

89. (canceled)

90. A system comprising:
a. a CRISPR effector protein or a polynucleotide encoding a CRISPR effector protein;
b. a guide polynucleotide comprising a guide sequence designed to hybridize with a target sequence in an MHC-associated gene in a cell; and
c. a donor polynucleotide comprising a polynucleotide sequence encoding a peptide for genetically modifying the MHC-associated gene via insertion of the donor polynucleotide at the MHC-associated gene in frame with the protein encoded by the MHC-associated gene, wherein the genetically modified MHC-associated gene encodes a fusion protein comprising the peptide.

91.-121. (canceled)
